(19) United States
(12) Patent Application Publication
(10) Pub. No.: US 2007/0088049 A1
(43) Pub. Date: Apr. 19, 2007

(54) POLYMORPHIC FORMS OF METHYL (+)-(S) - ALPHA - (2-CHLOROPHENYL)-6, 7-DIHYDROTHIENO [3,2-C]PYRIDINE-5(4H) ACETATE HYDROBROMIDE, CLOPIDOGREL HYDROBROMIDE

(75) Inventors: Keith Richard LORIMER, West Lafayette, IN (US); Alicia Tee Fuay Ng, West Lafayette, IN (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: Sanofi-aventis, Paris (FR)

(21) Appl. No.: 11/550,891

(22) Filed: Oct. 19, 2006

Related U.S. Application Data
(63) Continuation of application No. PCT/US05/13146, filed on Apr. 18, 2005.
(60) Provisional application No. 60/563,796, filed on Apr. 20, 2004.

(51) Int. Cl. A61K 31/4743; C07D 498/02

(52) U.S. Cl. 514/301; 546/114

(57) ABSTRACT

The invention relates to polymorphic Forms B, C, and D of methyl(+)-(S)-α-(2-chlorophenyl)-6,7-dihydrothieno[3,2-C]pyridine-5(4H) acetate hydrobromide, to pharmaceutical compositions containing the same, and to the method of use thereof for inhibiting platelet aggregation.

The invention relates to polymorphic Forms B, C, and D of methyl(+)-(S)-α-(2-chlorophenyl)-6,7-dihydrothieno[3,2-C]pyridine-5(4H) acetate hydrobromide, to pharmaceutical compositions containing the same and to the method of use thereof for inhibiting platelet aggregation. U.S. Pat. No. 4,847,265, issued Jul. 11, 1989, discloses the dextrorotatory enantiomer of methyl alpha-5-(4,5,6,7-tetrahydro-(3,2-C)thienopyridyl)(2-chlorophenyl)acetate or a pharmaceutically acceptable salt thereof. Specifically disclosed are the hydrochloride, hydrogen sulfate, hydrobromide, and taurocholate salts.

U.S. Pat. No. 6,429,210, issued Aug. 6, 2002, discloses polymorphic Form II of methyl(+)-(S)-α-(2-chlorophenyl)-6,7-dihydrothieno[3,2-C]pyridine-5(4H) acetate hydrogen sulfate known as clopidogrel hydrogen sulfate.

WO 03/066637, published Aug. 14, 2003, discloses crystalline Forms I and II of methyl-(S)-(+)-(2-chlorophenyl)-2-(6,7-dihydro-4H-thieno[3,2-C]pyridine-5-yl) acetate hydrochloride.

U.S. 2003/0114479, published Jun. 19, 2003, discloses crystalline Forms III, IV, and V, and an amorphous form of clopidogrel hydrogen sulfate.

U.S. 2003/0225129, published Dec. 4, 2003, discloses crystalline Forms III, IV, V, and VI and an amorphous form of clopidogrel hydrogen sulfate.

The solid state physical properties of a pharmaceutical compound can be influenced by the conditions under which the compound is obtained in solid form. Solid state physical properties include, for example, the flowability of the milled solid which affects the ease with which the compound is handled during processing into a pharmaceutical product. Another important solid state property of a pharmaceutical compound is its rate of dissolution in aqueous fluid. The rate of dissolution of an active ingredient in a patient's stomach fluid can have therapeutic consequences because it imposes an upper limit on the rate at which an orally administered active ingredient can reach the blood. The solid-state form of a compound may also affect its solubility, bioavailability, behavior on compaction, stability, or its electrostatic nature.

These physical properties of a pharmaceutical compound can be influenced by the conformation and orientation of molecules in the unit cell which defines a particular polymorphic form of a compound. The polymorphic form may give rise to thermal behavior different from that of the amorphous material or another polymorphic form. Thermal behavior is measured in the laboratory by such techniques as capillary melting point, thermogravimetric analysis and differential scanning calorimetry and can be used to distinguish one polymorphic form from another. A particular polymorphic form may also give rise to distinct properties that may be detectable by X-ray powder diffraction, solid-state ¹³CNMR spectrometry and infrared spectrometry.

The discovery or new crystalline polymorphic or amorphous forms of a pharmaceutical compound provides an opportunity to improve the physical or performance characteristics of a pharmaceutical product in that it enlarges the repertoire of materials that a formulation scientist has available for designing, for example, a pharmaceutical dosage form of a drug with a targeted release profile or other desired characteristic.

The invention relates to polymorphic Forms B, C, and D of methyl(+)-(S)-α-(2-chlorophenyl)-6,7-dihydrothieno[3,2-C]pyridine-5(4H) acetate hydrobromide of the formula I:

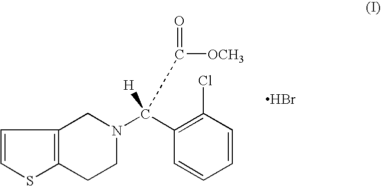

As described more particularly hereinafter, polymorphic Forms B, C and D of the present invention are distinguished from the hydrobromide salts disclosed in aforementioned U.S. Pat. No. 4,847,265.

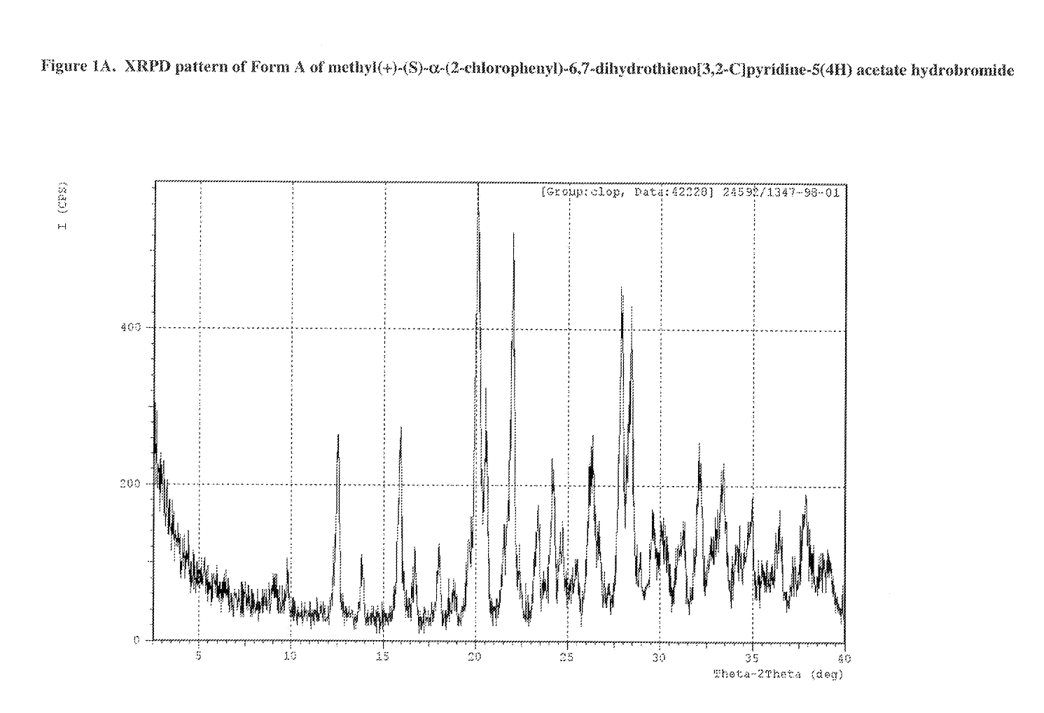

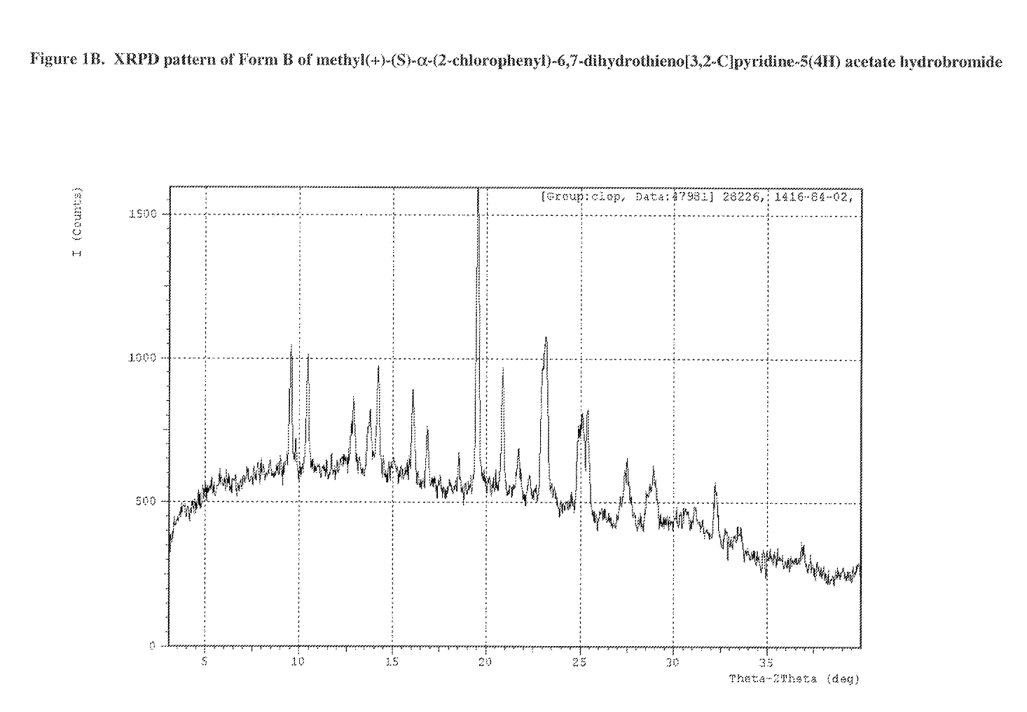

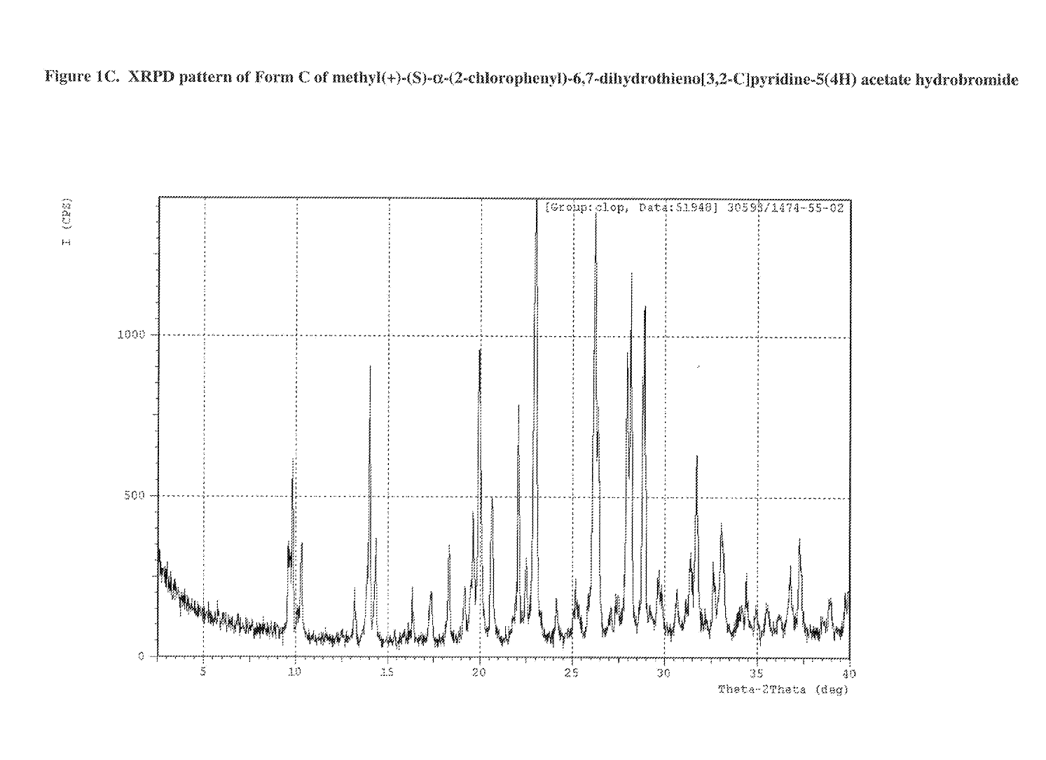

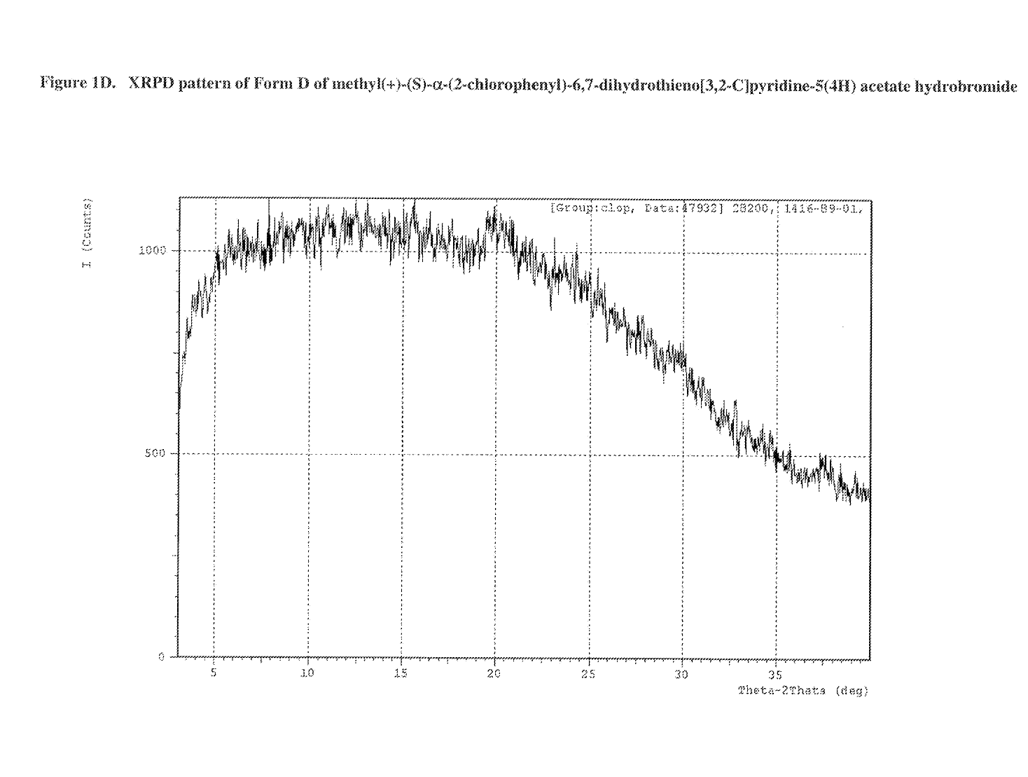

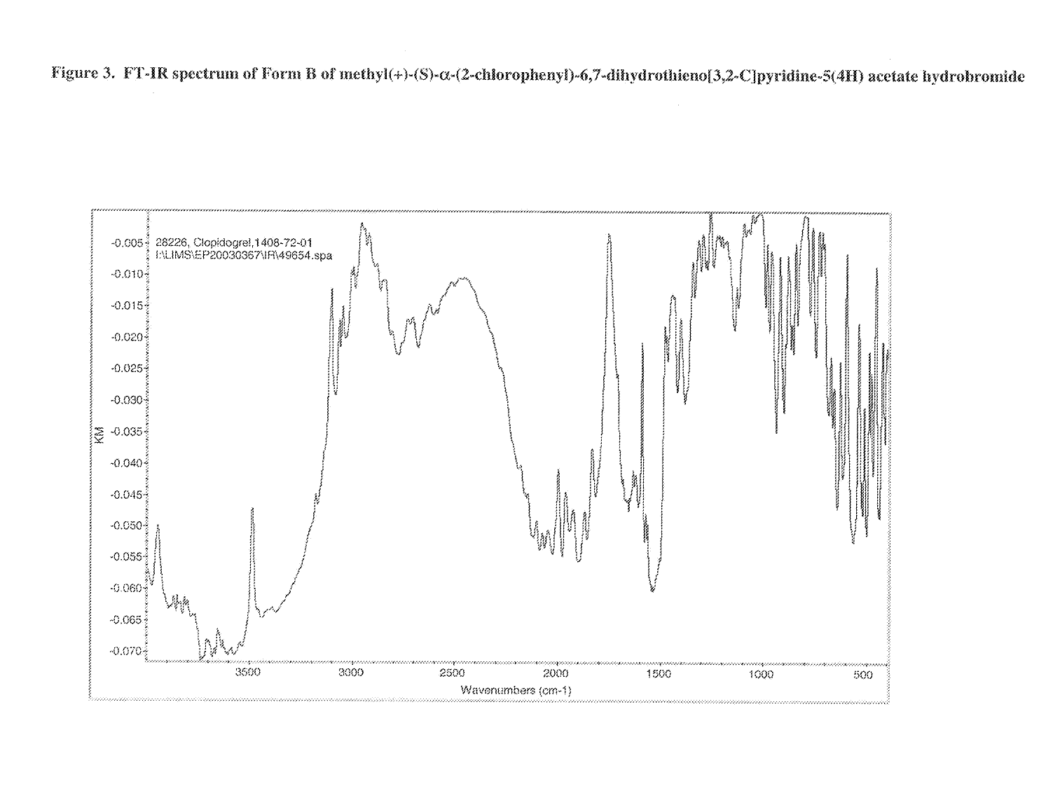

Polymorphic Form B is characterized by an X-ray powder diffraction pattern with a peak at about 20.9 degrees two-theta and more particularly with peaks at about 10.4, 14.2, 19.5 and 20.9 degrees two-theta. Form B is also characterized by an FTIR spectrum with peaks at about 537, 800, 1758, 3488, and 3949 cm⁻¹. Form B, which has a melting point of about 140-143°C., exhibits an X-ray powder diffraction pattern substantially as depicted in FIG. 1B and an FTIR spectrum substantially as depicted in FIG. 3.

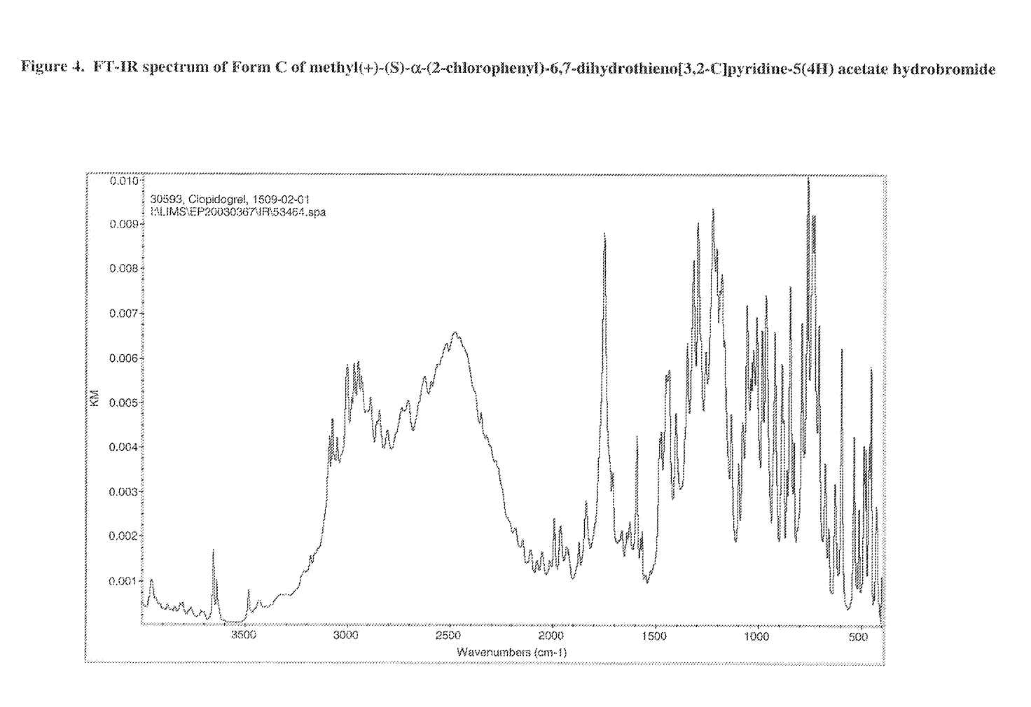

Polymorphic Form C is characterized by an X-ray powder diffraction pattern with a peak at about 22.0 degrees two-theta, and more particularly with peaks at about 20.6, 22.0, 28.1 and 31.7 degrees two-theta. Form C is also characterized by an FTIR spectrum with peaks at about 534, 789, 1753, 3639, 3657, and 3959 cm⁻¹. Form C, which has a melting point of about 138-148° C., exhibits an X-ray powder diffraction pattern substantially as depicted in FIG. 1C and an FTIR spectrum substantially as depicted in FIG. 4.

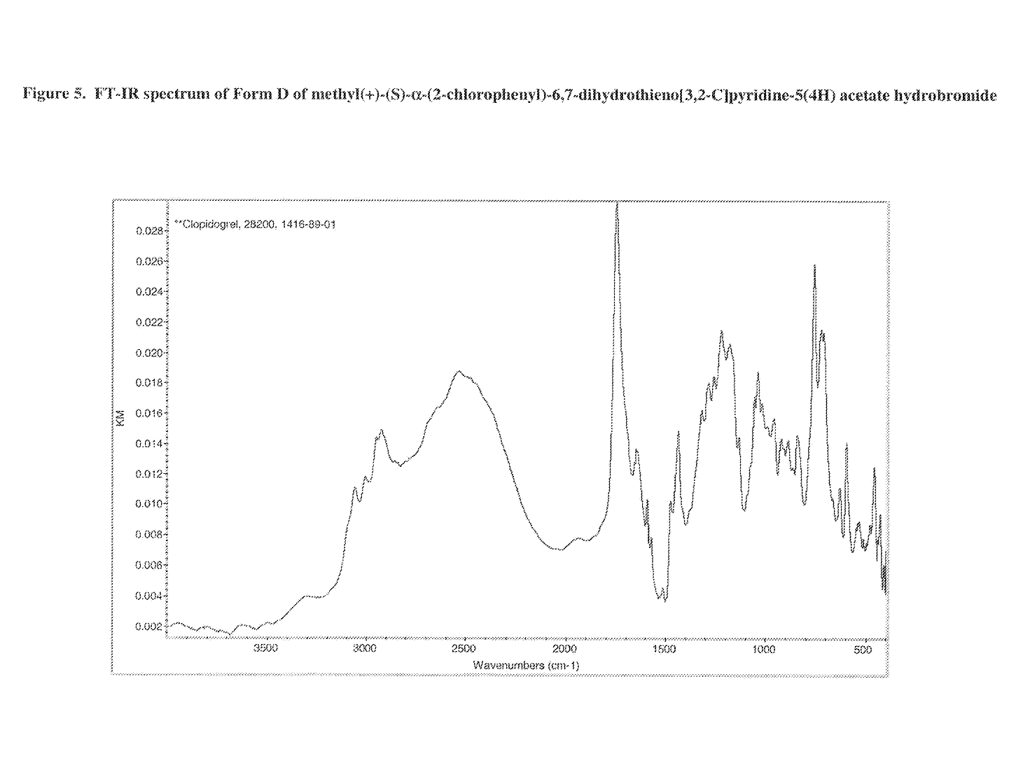

Plymorphic Form D is characterized by an FTIR spectrum with peaks at about 445, 723, 756, 1647, and 1748 cm⁻¹. Form D, exdhibits an X-ray powder dirffraction pattern substantially as depicted in FIG. 1D and an FTIR spectrum substantially as depicted in FIG. 5.

The present invention further relates to a pharmaceutical composition comprising: polymorphic Forms B, C, or D of methyl(+)-(S)-α-(2-chlorophenyl)-6,7-dihydrothieno[3,2-C]pyridine-5(4H) acetate hydrobromide, together with a pharmaceutically acceptable carrier, adjuvant, diluent, or vehicle.

The present invention further relates to a method for inhibiting platelet aggregation which comprises administering to a patient in need thereof an effective amount of polymorphic Form B, C, or D of methyl(+)-(S)-α-(2-chlorophenyl)-6,7-dihydrothieno[3,2-C]pyridine-5(4H) acetate hydrobromide.

The present invention further relates to the use of polymorphic Form B, C, or D of methyl(+)-(S)-α-(2-chlorophenyl)-6,7-dihydrothieno[3,2-C]pyridine-5(4H) acetate hydrobromide for the preparation of a medicament for inhibiting platelet aggregation.

The present invention further relates to a method of reducing atherosclerotic events which comprises administering to a patient in need thereof an effective amount of polymorphic Form B, C, or D of methyl(+)-(S)-α-(2-chlorophenyl)-6,7-dihydrothieno[3,2-C]pyridine-5(4H) acetate hydrobromide.

The present invention further relates to the use of polymorphic Form B, C, or D of methyl(+)-(S)-α-(2-chlorophenyl)-6,7-dihydrothieno[3,2-C]pyridine-5(4H) acetate hydrobromide for the preparation of a medicament for reducing atherosclerotic events.

FIG. 1A is an X-ray powder diffraction pattern of Form A of methyl(+)-(S)-α-(2-chlorophenyl)-6,7-dihydrothieno[3,2-C]pyridine-5(4H) acetate hydrobromide hydrate.

FIG. 1B is an X-ray powder diffraction pattern of Form B of methyl(+)-(S)-α-(2-chlorophenyl)-6,7-dihydrothieno[3,2-C]pyridine-5(4H) acetate hydrobromide.

FIG. 1C is an X-ray powder diffraction pattern of Form C of methyl(+)-(S)-α-(2-chlorophenyl)-6,7-dihydrothieno[3,2-C]pyridine-5(4H) acetate hydrobromide.

FIG. 1D is an X-ray powder diffraction pattern of Form D of methyl(+)-(S)-α-(2-chlorophenyl)-6,7-dihydrothieno[3,2-C]pyridine-5(4H) acetate hydrobromide.

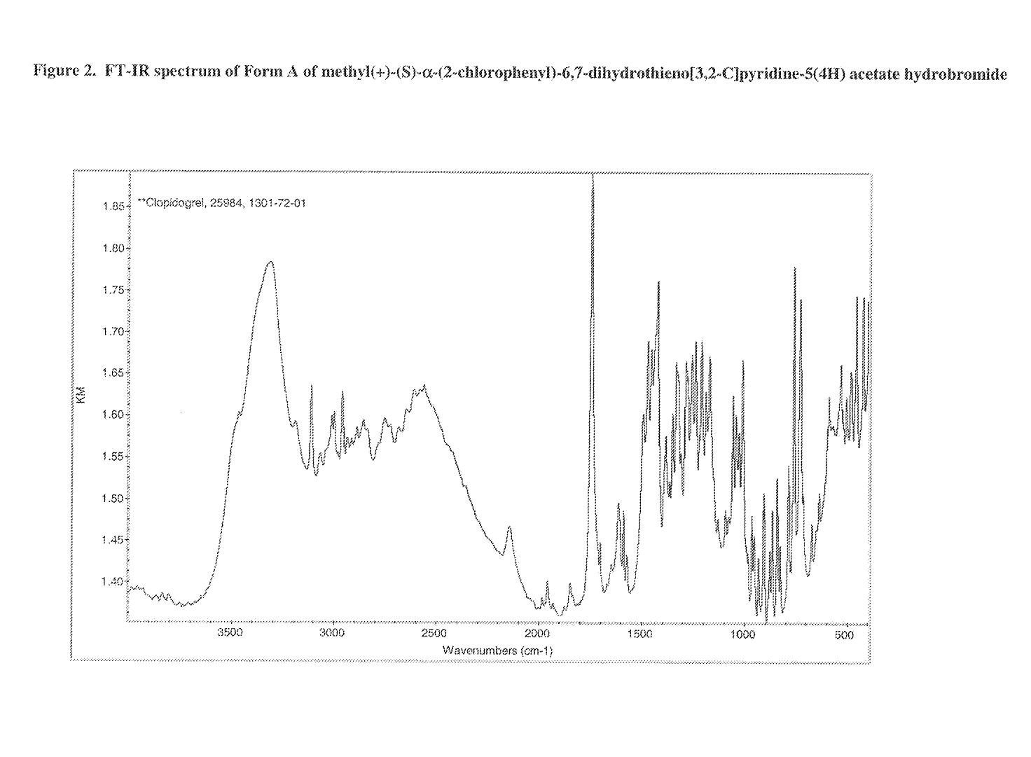

FIG. 2 is an FTIR spectrum of Form A of methyl(+)-(S)-α-(2-chlorophenyl)-6,7-dihydrothieno[3,2-C]pyridine-5(4H) acetate hydrobromide hydrate.

FIG. 3 is an FTIR spectrum of Form B of methyl(+)-(S)-α-(2-chlorophenyl)-6,7-dihydrothieno[3,2-C]pyridine-5(4H) acetate hydrobromide.

FIG. 4 is an FTIR spectrum of Form C of methyl(+)-(S)-α-(2-chlorophenyl)-6,7-dihydrothieno[3,2-C]pyridine-5(4H) acetate hydrobromide.

FIG. 5 is an FTIR spectrum of Form D of methyl(+)-(S)-α-(2-chlorophenyl)-6,7-dihydrothieno[3,2-C]pyridine-5(4H) acetate hydrobromide.

Form B of methyl(+)-(S)-α-(2-chlorophenyl)-6,7-dihydrothieno[3,2-C]pyridine-5(4H) acetate hydrobromide may be prepared by adding Form A of the compound to acetonitrile and then adding isopropylacetate to the solution until a precipitate of Form D is obtained. The solvents are decanted and evaporated to afford Form B.

Form C of methyl(+)-(S)-α-(2-chlorophenyl)-6,7-dihydrothieno[3,2-C]pyridine-5(4H) acetate hydrobromide may be prepared by dissolving Form A in a mixture of acetonitrile and isopropylacetate, seeding the solution with Form B, and then evaporating the solvents to afford Form C.

Form A of methyl(+)-(S)-α-(2-chlorophenyl)-6,7-dihydrothieno[3,2-C]pyridine-5(4H) acetate hydrobromide is obtained by reacting methyl(+)-(S)-α-(2-chlorophenyl)-6,7-dihydrothieno[3,2-C]pyridine-5(4H) acetate with hydrobromic acid as described in Example 1.

methyl(+)-(S)-α-(2-chlorophenyl)-6,7-dihydrothieno[3,2-C]pyridine-5(4H) acetate can be prepared, for example, by the method described in U.S. Pat. No. 4,847,265, which is incorporated herein by reference, or by the methods described herein in the examples.

The following examples will further illustrate the invention with, however, limiting it thereto. All melting points are given in degrees centigrade (° C.) and are obtained by placing the sample in a glass capillary. X-ray powder diffraction (XRPD) analyses were performed using a Shimadzu XRD-6000 (with a tube voltage of 40 kV, an amperage of 40 mA, divergence and scattering slits set at 1°, the receiving slit set at 0.15 mm, and a theta two theta continuous scan at 3°/min from 2.5 to 40° 2 theta) X-ray powder diffractometer using CuKα radiation. Infrared spectrum were acquired on a Magna-IR 860 Fourier transform infrared (FT-IR) spectrophotometer equipped with an Ever-Glo mid/far IR source, and the samples were prepared by mixing the sample with KBr.

### PREPARATION 1

### Methyl(+)-(S)-α-(2-chlorophenyl)-6,7-dihydrothieno[3,2-C]pyridine-5(4H) Acetate

A solution of clopidogrel hydrogensulfate (which can be prepared according to the methods described in U.S. Pat. No. 6,429,210 the contents of which are incorporated herein by reference) was treated with an aqueous solution of sodium carbonate. The title compound was extracted with diethyl ether and the solution was dried over MgSO₄ and the solvent was removed under reduced pressure to afford the title compound as a yellow gel.

### EXAMPLE 1

### Form A of methyl(+)-(S)-α-(2-chlorophenyl)-6,7-dihydrothieno[3,2-C]pyridine-5(4H) Acetate Hydrobromide Hydrate

A solution of methyl(+)-(S)-α-(2-chlorophenyl)-6,7-dihydrothieno[3,2-C]pyridine-5(4H) acetate of preparation 1 (1.8067 g in 25 mL of ethanol) (2.767 mL) was added to hydrobromic acid (2.0 mol/L, 0.310 mL). Heptane (1.00 mL) was added and the solution was filtered through a 0.2 μm nylon filter into a clean vial and left to evaporate under nitrogen. The solid which formed was slurried in a 1,4-dioxane-ethanol (9:1) mixture (1.0 mL) at room temperature and then the sample was then temperature cycled between 25-35° C. The sample was then refrigerated, filtered and dried to afford 0.0187 g of the title compound, m.p. 116° C. U.S. Pat. No. 4,847,265 discloses two hydrobromide salts, one melting at 111° C. and the other at 140° C. The compound of the instant example was analyzed by FTIR and XRPD and found to correspond to the lower melting hydrobromide salt disclosed in U.S. Pat. No. 4,847,265.

### EXAMPLE 2

### Form B of methyl(+)-(S)-α-(2-chlorophenyl)-6,7-dihydrothieno[3,2-C]pyridine-5(4H) Acetate Hydrobromide

Form A of methyl(+)-(S)-α-(2-chlorophenyl)-6,7-dihydrothieno[3,2-C]pyridine-5(4H) acetate hydrobromide hydrate of Example 1 (0.0323 g) was added to acetonitrile (0.200 mL) and the mixture was sonicated until complete dissolution was achieved. The solution was filtered through a 0.2 μm nylon filter into a clean vial and isopropylacetate (2.600 mL) was added until a precipitate formed. The solution was decanted off and was then filtered through a 0.2 μm nylon filter into a clean vial, and left to evaporate uncovered to dryness to afford the title compound, m.p. 140-143° C., which was analyzed by FTIR and XRPD.

### EXAMPLE 3

### Form C of methyl(+)-(S)-α-(2-chlorophenyl)-6,7-dihydrothieno[3,2-C]pyridine-5(4H) Acetate Hydrobromide

Form A of methyl(+)-(S)-α-(2-chlorophenyl)-6,7-dihydrothieno[3,2-C]pyridine-5(4H) acetate hydrobromide hydrate of Example 1 (0.1019 g) was dissolved in acetonitrile (0.500 mL) and isopropylacetate (1.0 mL) was added. Additional acetonitrile (0.10 mL) was added to the slightly murky solution. The solution was filtered through a 0.2 μm nylon filter into a clean vial and was seeded with a small amount of Form B of Example 2. The vial was covered with parafilm which was perforated with holes and the solution was left to evaporate to dryness to afford the title compound, m.p. 138-148° C., which was analyzed by FTIR and XRPD.

### EXAMPLE 4

### Form D of methyl(+)-(S)-α-(2-chlorophenyl)-6,7-dihydrothieno[3,2-C]pyridine-5(4H)

The precipitate obtain in Example 2 was dried to afford the title compound as an amorphous solid which was analyzed by FTIR and XRPD.

As disclosed in U.S. Pat. No. 4,847,265 and U.S. Pat. No. 5,576,328 (the entire contents of each of which is incorporated herein by reference) methyl(+)-(S)-α-(2-chlorophenyl)-6,7-dihydrothieno[3,2-C]pyridine-5(4H) acetate and its pharmaceutically acceptable salts have been found to possess valuable pharmacological properties. In particular, they have been found to inhibit platelet aggregation and thus would be useful in reducing atherosclerotic events, such as myocardial infarction, stroke, and vascular death.

The compounds of the invention are generally administered to patients which include, but are not limited to, mammals such as, for example, man. It will also be coadministered with other therapeutic or prophylactic agents and/or medicaments that are not medically incompatible therewith.

The compounds of the invention can be prepared for pharmaceutical use by conventional pharmaceutical procedures that are well known in the art, that is, by formulating a pharmaceutical composition which comprises compounds of the invention together with one or more pharmaceutically acceptable carriers, adjuvants, diluents or vehicles, for oral administration in solid or liquid form, parenteral administration, topical administration, rectal administration, or aerosol inhalation administration, and the like.

Solid compositions for oral administration include compressed tablets, pills, powders and granules. In such solid compositions, the active compound is admixed with at least one inert diluent such as starch, calcium carbonate, sucrose or lactose. These compositions may also contain additional substances other than inert diluents, e.g., lubricating agents, such as magnesium stearate, talc and the like.

Liquid compositions for oral administration include pharmaceutically acceptable emulsions, solutions, suspensions, syrups and elixirs containing inert diluents commonly used in the art, such was water and liquid paraffin. Besides inert diluents, such compositions may also contain adjuvants, such as, wetting and suspending agents and sweetening, flavoring, perfuming, and preserving agents. According to the invention, the compounds for oral administration also include capsules of absorbable material, such as gelatin, containing said active component with or without the addition of diluents or excipients.

Preparations according to the invention for parenteral administration include sterile aqueous, aqueous-organic, and organic solutions, suspensions and emulsions. Examples of organic solvents, or suspending media are propylene glycol, polyethylene glycol, vegetable oils such as olive oil and injectable organic esters such as ethyl oleate. These compositions can also contain adjuvants such as stabilizing, preserving, wetting, emulsifying and dispersing agents.

Preparations according to the invention for topical administration or aerosol inhalation administration include dissolving or suspending a compound of the invention in a pharmaceutically acceptable vehicle such as water, aqueous alcohol, glycol, oil solution or oil-water emulsion, and the like.

Prepartations according to the invention for rectal administration include suppositories prepared by using suitable carriers, e.g., cacao butter, hardened oils, glycerides or saturated fatty acids, and the like.

If desired, the compounds of the invention can further be incorporated into slow release or targeted delivery systems such as polymer matrices, liposomes, and microspheres.

The percentage of active component in such compositions may be varied so that a suitable dosage is obtained. The dosage administered to a particular patient is variable depending upon the clinician's judgment using as criteria: the route of administration, the duration of treatment, the size and physical condition of the patient, the potency of the active component, and the patient's response thereto. An effective dosage amount of the active component can thus readily by determined by the clinician after a consideration of all criteria and using his best judgment on the patient's behalf. In general, a compound of the instant invention is administered at a dose in the range of about 0.01 to about 100 mg/kg body weight.

What is claimed is:

1. Polymorphic Form B of methyl(+)-(S)-α-(2-chlorophenyl)-6,7-dihydrothieno[3,2-C]pyridine-5(4H) acetate hydrobromide.

2. Polymorphic Form B of methyl(+)-(S)-α-(2-chlorophenyl)-6,7-dihydrothieno[3,2-C]pyridine-5(4H) acetate hydrobromide according to claim 1 having an X-ray powder diffraction pattern with a peak at about 20.9 degrees two-theta.

3. Polymorphic Form B of methyl(+)-(S)-α-(2-chlorophenyl)-6,7-dihydrothieno[3,2-C]pyridine-5(4H) acetate hydrobromide according to claim 1 having an X-ray powder diffraction pattern with peaks at about 10.4, 14.2, 19.5 and 20.9 degrees two-theta.

4. Polymorphic Form B of methyl(+)-(S)-α-(2-chlorophenyl)-6,7-dihydrothieno[3,2-C]pyridine-5(4H) acetate hydrobromide according to claim 1 having an FTIR spectrum with peaks at about 537, 800, 1758, 3488, and 3949 cm⁻¹.

5. Polymorphic Form B of methyl(+)-(S)-α-(2-chlorophenyl)-6,7-dihydrothieno[3,2-C]pyridine-5(4H) acetate hydrobromide according to claim 1 having a melting point of about 140-143° C.

6. Polymorphic Form B of methyl(+)-(S)-α-(2-chlorophenyl)-6,7-dihydrothieno[3,2-C]pyridine-5(4H) acetate hydrobromide according to claim 1 having an X-ray powder diffraction pattern substantially as depicted in FIG. 1B.

7. Polymorphic Form B of methyl(+)-(S)-α-(2-chlorophenyl)-6,7-dihydrothieno[3,2-C]pyridine-5(4H) acetate hydrobromide according to claim 1 having an FTIR spectrum substantially as depicted in FIG. 3.

8. Polymorphic Form C of methyl(+)-(S)-α-(2-chlorophenyl)-6,7-dihydrothieno[3,2-C]pyridine-5(4H) acetate hydrobromide.

9. Polymorphic Form C of methyl(+)-(S)-α-(2-chlorophenyl)-6,7-dihydrothieno[3,2-C]pyridine-5(4H) acetate hydrobromide according to claim 8 having an X-ray powder diffraction pattern with a peak at about 22.0 degrees two-theta.

10. Polymorphic Form C of methyl(+)-(S)-α-(2-chlorophenyl)-6,7-dihydrothieno[3,2-C]pyridine-5(4H) acetate hydrobromide according to claim 8 having an X-ray powder diffraction pattern with peaks at about 20.6, 22.0, 28.1, and 31.7 degrees two-theta.

11. Polymorphic Form C of methyl(+)-(S)-α-(2-chlorophenyl)-6,7-dihydrothieno[3,2-C]pyridine-5(4H) acetate hydrobromide according to claim 8 having an FTIR spectrum with peaks at about 534, 789, 1753, 3639, 3657 and 3959 cm⁻¹.

12. Polymorphic Form C of methyl(+)-(S)-α-(2-chlorophenyl)-6,7-dihydrothieno[3,2-C]pyridine-5(4H) acetate hydrobromide according to claim 8 having a melting point of about 138-148° C.

13. Polymorphic Form C of methyl(+)-(S)-α-(2-chlorophenyl)-6,7-dihydrothieno[3,2-C]pyridine-5(4H) acetate hydrobromide according to claim 8 having an X-ray powder diffraction pattern substantially as depicted in FIG. 1C.

14. Polymorphic Form C of methyl(+)-(S)-α-(2-chlorophenyl)-6,7-dihydrothieno[3,2-C]pyridine-5(4H) acetate hydrobromide according to claim 8 having an FTIR spectrum substantially as depicted in FIG. 4.

15. Polymorphic Form D of methyl(+)-(S)-α-(2-chlorophenyl)-6,7-dihydrothieno[3,2-C]pyridine-5(4H) acetate hydrobromide.

16. Polymorphic Form D of methyl(+)-(S)-α-(2-chlorophenyl)-6,7-dihydrothieno[3,2-C]pyridine-5(4H) acetate hydrobromide according to claim 15 having an FTIR spectrum with peaks at about 456, 723, 756, 1647, and 1748 cm⁻¹.

17. Polymorphic Form D of methyl(+)-(S)-α-(2-chlorophenyl)-6,7-dihydrothieno[3,2-C]pyridine-5(4H) acetate hydrobromide according to claim 15 having an X-ray powder diffraction pattern substantially as depicted in FIG. 1D.

18. Polymorphic Form D of methyl(+)-(S)-α-(2-chlorophenyl)-6,7-dihydrothieno[3,2-C]pyridine-5(4H) acetate hydrobromide according to claim 15 having an FTIR spectrum substantially as depicted in FIG. 5.

19. A pharmaceutical composition comprising a compound according to claim 1 together with a pharmaceutically acceptable carrier, adjuvant, diluent, or vehicle.

20. A method for inhibiting platelet aggregation which comprises administering to a patient in need thereof an effective amount of a compound according to claim 1.

21. A method of reducing atherosclerotic events which comprises administering to a patient in need thereof an effective amount of a compound according to claim 1.
